(19) United States
(12) Patent Application Publication
(10) Pub. No.: US 2019/0151508 A1
(43) Pub. Date: May 23, 2019

(54) SYSTEM AND METHOD FOR EXTRACTING BIOLOGICAL MATERIALS FROM HARVESTED SKIN

(71) Applicant: Adam M. Rotunda, Irvine, CA (US)

(72) Inventor: Adam M. Rotunda, Irvine, CA (US)

(21) Appl. No.: 16/196,607

(22) Filed: Nov. 20, 2018

Related U.S. Application Data
(60) Provisional application No. 62/588,865, filed on Nov. 20, 2017.

(51) Int. Cl. A61L 27/36; C12N 5/077; C12N 5/071

(52) U.S. Cl. CPC A61L 27/3691; A61L 27/362; C12N 5/0625; C12N 5/0653

(57) ABSTRACT

Systems and methods for extracting and isolating components from a patient's dermis for autologous use are contemplated wherein dermal micrografts or microplugs are harvested from a patient via an arrayed transection process and subsequently homogenized and centrifuged. Such a process may permit skin components to be obtained in a minimally invasive and rapid fashion, including fat, collagen, fibroblasts, adipocytes, or plasma. For procedures in which only modest quantities of materials are required and benefits may be realized from using autologous materials, the disclosed techniques may be ideal.

### CROSS-REFERENCE TO RELATED APPLICATIONS

This application relates to and claims the benefit of U.S. Provisional Application No. 62/588,865 filed Nov. 20, 2017 and entitled “SYSTEM AND METHOD FOR EXTRACTING BIOLOGICAL MATERIALS FROM HARVESTED SKIN,” the entire disclosure of which is hereby wholly incorporated by reference.

### STATEMENT RE: FEDERALLY SPONSORED RESEARCH/DEVELOPMENT

Not Applicable

### BACKGROUND

### 1. Technical Field

The present disclosure relates generally to systems and methods for extracting autologous fat, collagen, and platelet-rich plasma (PRP) from skin. More particularly, the present disclosure relates to systems and methods for extracting micrografts or microplugs from skin and processing these extracted micrografts or microplugs into useful biological materials.

### 2. Related Art

There exists great interest in the fields of medical, reconstructive, and cosmetic surgery in the potential applications of autologous transplantation. For example, PRP is a clinical tool that utilized in many types of therapies. PRP is typically formed by extracting a volume of patient's blood, adding an anticoagulation agent, centrifuging that blood to remove red blood cells, and possibly taking further action to enrich the concentration of platelets and/or other remaining components within the plasma. While, the circulating blood of a healthy patient typically contains about two hundred million platelets per mL, FDA-approved methods for forming PRP concentrate generally result in about a five-fold increases in platelet concentration, to around one billion platelets per mL. Additionally, the process for forming PRP may increase the concentration of the circulating growth factors, cytokines, and leukocytes above the levels found in whole blood.

However, a number of deficiencies and misunderstandings currently exist in the field of PRP-based therapies, especially among therapies involving autologous grafting or healing of surgical wounds. An uptick in media coverage fueled by endorsement by professional athletes of various PRP-based therapies of questionable benefit has exacerbated this confusion, often causing medical practitioners and the public at large to misunderstand the benefits of PRP therapies, and to attribute a panacea of benefits to the basic therapy of injecting PRP by itself, when often such therapies may have little or no clinical efficacy.

Therapies involving the transplantation of collagen are also finding growing acceptance. Where previously most collagen-based therapies have involved the use of hydrolyzed porcine or bovine collagen as an injectable filler or as a component in a topically-applied cream for cosmetic or reconstructive purposes, autologous collagen may have promise as a material superior to bovine or porcine collagen in many medical applications. For example, the recently developed arthroscopic cartilage repair treatment known as autologous collagen-induced chondrogensis (ACIC) utilizes a low-immunogenic derivative of animal collagen obtained by removal of N- and C-terminal telopeptide components (atelocollagen) as a scaffolding material for the implantation of human mesenchymal stem cells cultured in chondrogenic differentiation medium. It may be seen that if autologous collagen were utilized rather than low-immunogenic processed animal collagen, any immunogenic risk may be eliminated.

Autologous fat grafting is another emerging technique with medical, reconstructive, and cosmetic applications. For example, breast reconstruction following mastectomy or lumpectomy may be performed via autologous fat transfer, typically harvested via liposuction. Autologous fat grafting has also been used during or following cosmetic or medical midfacial surgeries, such as rhytidectomy or blepharoplasty. However, liposuction may not be a desirable method in all circumstances for harvesting autologous fat.

Thus, there exists a need in the art for a system and method for extracting biological materials such as PRP precursors, collagen, and fat.

### BRIEF SUMMARY

To solve these and other problems, systems and methods for specifically address and alleviates the above-identified deficiencies in the art are contemplated.

According to one embodiment of the contemplated disclosure, a method of extracting and isolating fat from a patient's dermis for autologous use is contemplated as comprising the steps of harvesting a plurality of dermal grafts from a patient, the plurality of dermal grafts being obtained via an arrayed transection process, homogenizing the dermal grafts, centrifuging the homogenized dermal grafts, and isolated the fat component of the centrifuged, homogenized dermal grafts.

The harvesting step may be performed via an arrayed transection process operative to produce a plurality of elliptical dermal grafts, the mean diameter of the respective ones of the plurality of elliptical dermal grafts being between ½ mm and 1 mm. The elliptical dermal grafts may also be circular.

The harvesting step may also be performed via an arrayed transection process operative to produce a plurality of quadrilateral dermal grafts, the mean length of the smallest side of the respective ones of the plurality of quadrilateral dermal grafts being less than 1 mm. The quadrilateral dermal grafts may be square.

According to other contemplated embodiments, other components for autologous use may be extracted and isolated from the harvested dermal grafts via a similar process, such as collagen, fibroblasts, adipocytes, and plasma.

### DETAILED DESCRIPTION

According to various aspects of the present disclosure, methods and compositions for harvesting components from dermal micrografts and microplugs are contemplated. Specifically, it is contemplated that dermal micrografts and microplugs may serve as ready sources of autologous materials for use in autologous grafts. In an exemplary embodiment, an arrayed transection process may be utilized to harvest a plurality of micro (<1 mm) grafts or plugs from a section of a patient's skin, which are subsequently homogenized and centrifuged to permit isolation of their constituents for use in autologous grafting processes. Many benefits may be derived from the use of these micrograft or microplug derived skin constituents for autologous grafts. For example, it may be seen that this process may elimination of any potential for immunogenic response or rejection relative to conventional sources of these skin constituents, such as porcine or bovine sources. It may also be seen that the relative availability and non-invasiveness of a skin micrograft or microplug harvesting technique relative to conventional sources of autologous materials, which may require invasive surgery such as liposuction, may represent a substantial benefit as well, as it may not be suitable or desirable to obtain these materials through a more invasive procedure when only a modest quantity of the required material may be required.

Various transection processes are known in the art for extracting a quantity of micrografts or microplugs from a patient's dermis. Most commonly, such techniques are used in the context of follicular unit transplantation or follicular unit extraction, whereby individual hair follicles, or groups of hair follicles in strips, are transplanted from one location on a patient's body to another.

In other contexts, micrograft or microplug extraction is utilized for purposes of skin tightening. To this end, various micrograft or microplug harvesting process have been developed. For example, the process discussed in U.S. Pat. No. 8,900,181 relates to the use of an array of scalplets positioned on a rollable surface to create an array of spaced micro-incisions of between ½ to 1 millimeter in size on a patient's dermis, with that array of spaced incisions subsequently being harvested via transection. The arrayed harvesting of the micrografts or microplugs in this fashion in combination with the application of a compressive sheet or bandage to vector and contour during the wound healing process may permit a skin tightening process to be performed in the aggregate rather than via a substantial resection of a skin flap, which may leave substantially less scarring and may result in a more preferred aesthetic outcome. Additionally, the process discussed in WO2009146068 relates to the insertion of an array of thin, hollow tubes of around 0.3 to 1 millimeter in diameter at various angles into the dermis, which may permit the arrayed harvesting of thin micrograft strips or microplugs from the patient, depending at the angle of insertion. Such micrografts are envisioned therein as being useful for promoting better wound healing, especially in small, well-vascularized wounds, or with those micrografts being formed into a meshed aggregate as a substitute for more conventional large scale grafts full-thickness grafts, which may be of benefit to children due to a greater tolerance and capability for future growth relative to conventional bulk grafts.

While various of these techniques for arrayed micrograft/microplug harvesting are known, these micrografts and microplugs have only been utilized for direct transplantation, whereby the micrograft or microplug is reinserted into another portion of the patient's skin in relatively the same structural orientation and configuration as the graft had when it was removed. The potential for use of these micrografts or microplugs as a source of component materials, which may be used in all forms of autologous grafting, and not merely skin transplantation, is relatively unexplored.

In this regard, it is contemplated that it may be desirable to first harvest of a plurality of dermal grafts from a patient may via an arrayed transection process, whereby the dermal micrografts or microplugs are directly transected from the epidermis in an arrayed process (as opposed to the formation of the micrografts or microplugs via a process of subdividing or otherwise cutting up a previously transected bulk graft). The use of an arrayed transection process may permit a minimal amount of invasiveness, and a greater convenience to the patient, especially as the actual process of harvesting via transection may not be the intended goal in and of itself, combined the undesirability of potentially leaving any permanent scarring.

Following the harvesting process, the harvested dermal grafts are homogenized, which will result in a resulting material of uniform composition, which may result in the formation of a material which may be a liquid or a very fine solid, and preferably is a suspension. It may be desirable before, during or after the homogenization process to remove undesirable components which may have been included in the harvested dermal micrografts or microplugs, such as foreign bodies, skin cysts, epidermal inclusions, adnexal structures (such as hair, eccrine, or apocrine glands). Such removal may occur prior to the harvesting process as well, such as via application of light or laser energy, or following the harvesting process but before the homogenization process, such as via mechanical removal of an epidermal inclusion, or following the homogenization process, such as via a filtering, which may remove dense foreign bodies or skin cysts. It may also be appreciated that the homogenization process itself may be performed via a mechanical process, such as via blending, chopping, or grinding, or via a chemical process, such as application of a detergent, or surfactant, or via any other process by which homogenization may occur, such as via the application of sonic energy, or via combinations of one or more of these processes. It may also be appreciated that the technique of homogenization may be tailored or chosen according to the specific component or component desired to be extracted from the dermal micrografts or microplugs. For example, it if may be desired to extract intact cells, such as fibroblasts, it will be preferred that a homogenization technique is utilized which does not result in the general lysing of cells, with such general lysing techniques including certain chemical homogenization techniques or mechanical techniques which shear the cell membrane. Conversely, if it may be desired to obtain certain intracellular components, it may be desirable to utilize one of these cell lysing homogenization techniques. Likewise, if it is desirable to obtain, for example, collagen, it may be desirable to use a homogenization technique which does not disrupt the self-assembled fibrillar collagen structure.

It is also contemplated that as an alternative to direct autologous placement of harvested collagen or fat, the herein discussed harvesting, homogenization, and isolation techniques may also be applicable to isolate fibroblasts or adipocytes, or another specific desired cellular components of the patient's dermis. Once isolated, this desired cellular component of the dermis may be cloned and cultured to produce a stable and non-immunogenic final biological product, such as collagen or fat. After processing and purification, the final biologic product, which may be collagen or fat, is reinjected into the patient from which the grafts were obtained. In this fashion, it may be seen that a substantial amount of the specific desired cellular components of the dermis may also be obtained for an autologous purpose via the herein discussed arrayed transection process.

Following the homogenization process, the homogenized dermal grafts are centrifuged in order to separate the individual component of the homogenized dermal grafts from one another in order to permit isolation. It may be appreciated that the exact parameters of the centrifugation process may vary according to the specific component or components targeted for isolation, as well as other variables unique to the patient or the source of the grafts on the patient's body. However, it may be seen that following centrifugation, the individual components of the homogenized dermal grafts will be linearly separated and be suitable for isolation via known techniques of isolating a centrifuged composition. Such isolated compositions which are readily present in dermal micrografts or microplugs may be, for example but without limitation, fat, collagen, fibroblasts, and plasma.

Following isolation, the isolated components may be immediately suitable for autologous use, as they will be completely non-immunogenic for the patient from which they were extracted. In this way, it may be seen that an autologous preparation may be prepared in a relative rapid timeframe without requiring any invasive procedure, and by nature of being autologous, risk of immune response or other rejection will be minimized. As such, for procedures in which only a modest quantity of autologous materials is required, including but not limited to topical or cutaneous applications for the skin, hair or nail, soft tissue fillings, and topical application or injections for wound healing purposes, it may be seen that a method according to the herein discussion may be highly suitable and highly desirable. Furthermore, it may be seen that devices may be utilized which may be suitable not only to perform the harvesting steps, but such devices may also have integrated within them the means to perform the herein discussed homogenization, centrifugation, and/or extraction steps as well, as well as any further intermediate or subsequent processing steps which may be desired, such as a processing step for converting harvested and isolated plasma to platelet-rich plasma.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the exemplary embodiments.

What is claimed is:

1. A method of extracting and isolating fat from a patient's dermis for autologous use, the method comprising the steps of:
harvesting a plurality of dermal grafts from a patient, the plurality of dermal grafts being obtained via an arrayed transection process;
homogenizing the dermal grafts;
centrifuging the homogenized dermal grafts; and
isolating the fat component of the centrifuged, homogenized dermal grafts.

2. The method of claim 1, wherein the harvesting step is performed via an arrayed transection process operative to produce a plurality of elliptical dermal grafts, the mean diameter of the respective ones of the plurality of elliptical dermal grafts being between ½ mm and 1 mm.

3. The method of claim 2 wherein the elliptical dermal grafts are circular.

4. The method of claim 1, wherein the harvesting step is performed via an arrayed transection process operative to produce a plurality of quadrilateral dermal grafts, the mean length of the smallest side of the respective ones of the plurality of quadrilateral dermal grafts being less than 1 mm.

5. The method of claim 4, wherein the quadrilateral dermal grafts are square.

6. A method of extracting and isolating collagen from a patient's dermal for autologous use, the method comprising the steps of:
harvesting a plurality of dermal grafts from a patient, the plurality of dermal grafts being obtained via an arrayed transection process;
homogenizing the dermal grafts;
centrifuging the homogenized dermal grafts; and
isolating the collagen component of the centrifuged, homogenized dermal grafts.

7. The method of claim 6, wherein the harvesting step is performed via an arrayed transection process operative to produce a plurality of elliptical dermal grafts, the mean diameter of the respective ones of the plurality of elliptical dermal grafts being between ½ mm and 1 mm.

8. The method of claim 7 wherein the elliptical dermal grafts are circular.

9. The method of claim 6, wherein the harvesting step is performed via an arrayed transection process operative to produce a plurality of quadrilateral dermal grafts, the mean length of the smallest side of the respective ones of the plurality of quadrilateral dermal grafts being less than 1 mm.

10. The method of claim 9, wherein the quadrilateral dermal grafts are square.

11. A method of extracting and isolating plasma from a patient's dermis for autologous use, the method comprising the steps of:
harvesting a plurality of dermal grafts from a patient, the plurality of dermal grafts being obtained via an arrayed transection process;
homogenizing the dermal grafts; or extracting the requisite fluid (plasma) for cloning and purification into an autologous collagen product;
centrifuging the homogenized dermal grafts; and
isolating the plasma component from the centrifuged, homogenized dermal grafts.

12. The method of claim 11, wherein the harvesting step is performed via an arrayed transection process operative to produce a plurality of elliptical dermal grafts, the mean diameter of the respective ones of the plurality of elliptical dermal grafts being between ½ mm and 1 mm.

13. The method of claim 12 wherein the elliptical dermal grafts are circular.

14. The method of claim 11, wherein the harvesting step is performed via an arrayed transection process operative to produce a plurality of quadrilateral dermal grafts, the mean length of the smallest side of the respective ones of the plurality of quadrilateral dermal grafts being less than 1 mm.

15. The method of claim 14, wherein the quadrilateral dermal grafts are square.

16. A method of extracting and isolating a desired cellular component from a patient's dermis for autologous use, the method comprising the steps of:
harvesting a plurality of dermal grafts from a patient, the plurality of dermal grafts being obtained via an arrayed transection process;
homogenizing the dermal grafts;
centrifuging the homogenized dermal grafts; and
isolating the desired cellular component of the centrifuged, homogenized dermal grafts.

17. The method of claim 16, wherein the harvesting step is performed via an arrayed transection process operative to produce a plurality of elliptical dermal grafts, the mean diameter of the respective ones of the plurality of elliptical dermal grafts being between ½ mm and 1 mm.

18. The method of claim 17, wherein the elliptical dermal grafts are circular.

19. The method of claim 16, wherein the harvesting step is performed via an arrayed transection process operative to produce a plurality of quadrilateral dermal grafts, the mean length of the smallest side of the respective ones of the plurality of quadrilateral dermal grafts being less than 1 mm.

20. The method of claim 19, wherein the quadrilateral dermal grafts are square.

21. The method of claim 16, wherein the desired cellular component comprises one or more of: fibroblasts, adipocytes, mesenchymal stem cells, chondrocytes.

22. The method of claim 21, wherein the isolated desired cellular component comprises fibroblasts, and the fibroblasts are cultured to produce non-immunogenic collagen for administration to the patient.

23. The method of claim 21, wherein the isolated desired cellular component comprises adipocytes, and the adipocytes are cultured to produce non-immunogenic fat for administration to the patient.
